(19) United States
(12) Patent Application Publication
(10) Pub. No.: US 2010/0197610 A1
(43) Pub. Date: Aug. 5, 2010

(54) FRUCTOSE 1, 6 BISPHOSPHATE - A NOVEL ANTICONVULSANT DRUG

(75) Inventors: Xiao-Yuan Lian, Houston, TX (US); Janet L. Stringer, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX (US)

(21) Appl. No.: 12/523,882

(22) PCT Filed: Jan. 23, 2008
(86) PCT No.: PCT/US08/51777

§ 371 Date: Mar. 8, 2010

Related U.S. Application Data
(60) Provisional application No. 60/886,163, filed on Jan. 23, 2007.

(51) Int. Cl. A61K 31/70; A61P 25/08

(52) U.S. Cl. 514/23

(57) ABSTRACT

The present invention concerns methods and compositions for preventing one or more epileptic seizures in an individual by delivering fructose-1,6-bisphosphate to the individual. In certain cases, an additional therapy for epilepsy is provided to the individual.

### CROSS-REFERENCE TO RELATED APPLICATIONS

This application claims priority to U.S. Provisional Patent Application No. 60/886,163, filed on Jan. 23, 2007, which is incorporated by reference herein in its entirety.

### STATEMENT REGARDING FEDERALLY SPONSORED RESEARCH OR DEVELOPMENT

This invention was made with government support under National Institute of Health Grant No. NS039941. The government has certain rights in the invention.

### TECHNICAL FIELD

The present invention concerns at least the fields of medicine, cell biology, physiology, pharmacology, biochemistry, neuroscience, and molecular biology. In particular, the present invention concerns the field of epilepsy.

### BACKGROUND OF THE INVENTION

Glucose is the primary source of energy for the central nervous system. Imaging of children with Lennox-Gastaut and infantile spasms has shown decreased glucose utilization between seizures and excessive glycolysis immediately prior to, and during, seizures (Chugani and Chugani, 2003). In addition, a cerebral deficit in the reduced form of glutathione (GSH), which is an important free radical scavenger in the mammalian nervous system (Wu et al., 2004) and an endogenous anticonvulsant (Abe et al., 2000), has been shown in patients with partial seizures (Mueller et al., 2001). Oxidized glutathione is reduced by NADPH generated in the pentose phosphate pathway. The pentose phosphate pathway is an alternative pathway for glucose metabolism that generates NADPH for use in reductive biosynthesis.

Evidence indicates that the changes in glucose metabolism and decreased glutathione levels observed in the brains of patients with epilepsy favor the generation of each seizure. First, hyperglycemia has been associated with seizure activity (Schwechter et al., 2003; Lammouchi et al., 2004), while relative hypoglycemia has been shown to have an anticonvulsant effect (Greene et al., 2001). Second, the ketogenic diet, which provides energy substrates for the brain that bypass glycolysis, has been shown to be an effective treatment for seizures (Freeman et al., 2007). Finally, animals with low levels of GSH have a low seizure threshold or spontaneous seizures (Wu et al., 2004).

Fructose-1,6-bisphosphate (F1,6BP) has actions that suggest it may be an effective anticonvulsant (FIG. 1). First, F1,6BP has been shown to increase flux of glucose into the pentose phosphate pathway (Kelleher et al., 1995; Espanol et al., 1998) and preserve cellular GSH levels (Vexler et al., 2003). Second, F1,6BP modulates the activity of phosphofructokinase-1 (PFK-1), which is the enzyme that controls the rate-limiting step in glycolysis. F1,6BP is a weak stimulator of PFK-1, but becomes inhibitory in the presence of fructose-2,6-bisphosphate (F2,6BP), a potent activator of PFK-1 (Van Schaftingen, 1987; Heylen et al., 1982). These data indicate that F1,6BP will slightly enhance basal glucose metabolism, but will prevent stimulation of glycolysis by F2,6BP. Diverting glucose from glycolysis towards the pentose phosphate pathway, thus increasing GSH levels while maintaining an energy source for the brain, should provide significant anticonvulsant efficacy.

The mechanism of action of F1,6BP has been debated, in part, because of the general belief that charged, phosphorylated sugars cannot cross cell membranes, particularly the blood brain barrier. However, it has been shown that FDP is capable of entering cells and serving as a glycolytic intermediate. This was done with ¹³C-labeled FDP in smooth muscle cells from pig artery in vitro (Hardin and Roberts, 2004). FDP has also been shown to diffuse across a membrane bilayer in a dose-dependent fashion (Ehringer et al., 2000). The same study also showed dose-dependent uptake of ¹⁴C-FDP into endothelial cells. The data indicate that FDP crosses the membrane intact.

Recently, it has been shown that exogenous administration of FDP can reduce the duration and severity of seizures in laboratory animals (Lian et al., 2007). In these studies, the FDP was given into the peritoneal cavity. Despite the clear effect of FDP on seizure activity, the question remains whether FDP can get into the brain. In earlier experiments it was shown that administration of FDP to rabbits during hypoglycemic coma (Farias et al., 1989) or ischemia-hypoxia and reperfusion (Farias et al., 1990) improved outcomes. Experiments have also shown alterations in pyruvate levels in the brain of pigs after intravenous administration of FDP (Kaakinen et al., 2006). Finally, exogenous administration has been shown to have neuroprotective activity in pigs (Kaakinen et al., 2005) and mice (Rogido et al., 2003).

The present invention provides a novel solution for a long-felt need in the art for an alternative to known therapies to prevent epileptic seizures.

### BRIEF SUMMARY OF THE INVENTION

The present invention is directed to methods and compositions that relate to epilepsy. In certain embodiments, the present invention concerns prevention and/or treatment of epilepsy. In further embodiments, the present invention concerns prevention and/or treatment of symptoms of epilepsy, including seizure.

In some embodiments of the invention, there is a method of preventing one or more epileptic seizures in an individual with epilepsy by delivering to the individual a therapeutically effective amount of fructose-1,6-bisphosphate (which may also be referred to as fructose 1,6 diphosphate (FDP)). The term “preventing” as used herein refers to completely inhibiting an epileptic seizure, delaying onset of an epileptic seizure, reducing frequency of epileptic seizures, reducing length and/or intensity of an epileptic seizure, or delaying onset and/or reducing frequency and/or reducing length and/or reducing intensity of an epileptic seizure.

In a specific embodiment, the individual is delivered fructose-1,6-bisphosphate in any suitable administration route and regimen such that it results in prevention of one or more epileptic seizures. In further specific embodiments, the individual is delivered fructose-1,6-bisphosphate at a dosage suitable to prevent one or more epileptic seizures. In particular cases, the dosage of fructose-1,6-bisphosphate is 50-150 mg/kg. In certain embodiments, the individual is provided multiple deliveries of fructose-1,6-bisphosphate, although in alternative embodiments the individual is provided a single delivery of fructose-1,6-bisphosphate to prevent at least one epileptic seizure. In a specific embodiment, multiple deliveries occur from 12 hours to six days apart. In specific embodiments, a derivative of fructose-1,6-bisphosphate is employed in the invention, for example, 2,5-anhydromannitol.

In some embodiments, the individual is provided fructose-1,6-bisphosphate when the individual is suspected of having epilepsy, at high risk for developing epilepsy, or when the individual is known to have epilepsy. An individual suspected of having epilepsy may be an individual that has had one or two seizures. An individual at risk for developing epilepsy is one having family history (and, in some cases, may be genetically predisposed to epilepsy, such as having a mutation in SCN2A, for example (Bergren et al., 2005)); one having had a brain insult, including a brain injury, stroke, or surgery; one having a brain tumor; one having intolerance to wheat; one exposed to high levels of lead; one that has hypoglycemia, one that has hypoxia, and/or one that has used recreational drugs.

In some cases, the method further comprises delivering an additional therapy for epilepsy to the individual. In some cases, the additional therapy is a drug, vagus nerve stimulation, surgery, dietary therapy, or a combination thereof. In specific embodiments, the drug is selected from the group consisting of carbamazepine, Carbatrol®, Clobazam, Clonazepam, Depakene®, Depakote®, Depakote ER®, Diastat, Dilantin®, Felbatol®, Frisium, Gabapentin®, Gabitril®, Inovelon®, Keppra®, Klonopin, Lamictal®, Lyrica, Mysoline®, Neurontin®, Oxcarbazepine, Phenobarbital, Phenylek®, Phenyloin, Rufinamide, Sabril, Tegretol®, Tegretol XR®, Topamax®, Trileptal®, Valproic Acid, Zarontin®, Zonegran, and Zonisamide.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 provides a schematic illustration of glucose utilization through the glycolytic and the pentose phosphate pathways. The sites of action for F1,6BP are indicated. Abbreviations: F1,6BP, fructose-1,6-bisphosphate; P, phosphate; PFK, phosphofructokinase; PPP, the pentose phosphate pathway, (+)=stimulatory activity to the pathway or the enzyme; (−)=inhibitory activity.

FIG. 2 shows an anticonvulsant effect of F1,6BP in the pilocarpine model. One hour before the pilocarpine (300 mg/kg) animals received one of the following: saline (as seizure controls, Pilo), F1,6BP (0.25, 0.5 or 1 g/kg, pre-F1,6BP), F1,6BP (1 g/kg) plus lactate (0.5 g/kg) (F1,6BP/Lac), 2-DG (0.25 g/kg), 2-DG (0.25 g/kg) plus lactate (0.5 g/kg) (2-DG/Lac), VPA (0.3 g/kg), ketogenic diet (starting at 20 days old, KD-Yng; or at 2 months of age, KD-Adult). Some animals received F1,6BP after the first behavioral seizure (post-F1,6BP). In A, the mean (±SEM) for each measured seizure parameter is shown for each treatment group. *=p<0.05, **=p<0.01 compared to Pilo; #=p<0.05, ##=p<0.01 vs VPA.

FIG. 3 demonstrates an anticonvulsant effect of F1,6BP in the kainic acid model. One hour before the kainic acid (10 mg/kg) animals received one of the following: saline (as seizure controls, KA), F1,6BP (0.5 or 1 g/kg), 2-DG (0.25 g/kg), VPA (0.3 g/kg), ketogenic diet (starting at 20 days old, KD-Yng; or at 2 months of age, KD-Adult). The mean (±SEM) for each measured seizure parameter is shown for each treatment group. * p<0.05, ** p<0.01 vs KA. # p<0.05, ##<0.01 vs VPA.

FIG. 4 shows an anticonvulsant effect of F1,6BP in the PTZ model. One hour before PTZ (50 mg/kg) animals received one of the following: saline (as seizure controls, PTZ), F1,6BP (0.25, 0.5 or 1 g/kg), 2-DG (0.25 or 0.5 g/kg), VPA (0.3 g/kg). The mean (±SEM) for each measured seizure parameter is shown for each treatment group. * p<0.05, ** p<0.01 compared to PTZ.

FIG. 5 demonstrates kinetics of fructose 1,6-diphosphate (FDP) after intraperitoneal administration. Levels of FDP in whole blood (top) and brain (bottom) are presented as a function of time after administration of a single dose of 0.5 g/kg. Each point represents the mean±SEM and the number of animals in each group is indicated beside each point. The asterisk indicates a significant difference compared to the control values, which are combined vehicle control animals (n=3) and naïve animals (n=9).

FIG. 6 provides exemplary levels of fructose 1,6-diphosphate (FDP) in peripheral tissues. Levels of FDP were determined in 4 additional tissues in naïve animals and animals treated with a single dose of 0.5 g/kg FDP then sacrificed at either 1 or 12 hours. There were 6 samples in each tissue group. A 1-way ANOVA was used to compare the levels within each tissue and only the 1 hour samples were significantly different from control in muscle and fat.

FIG. 7 illustrates exemplary anticonvulsant action of oral administration of fructose-1,6-bisphosphate.

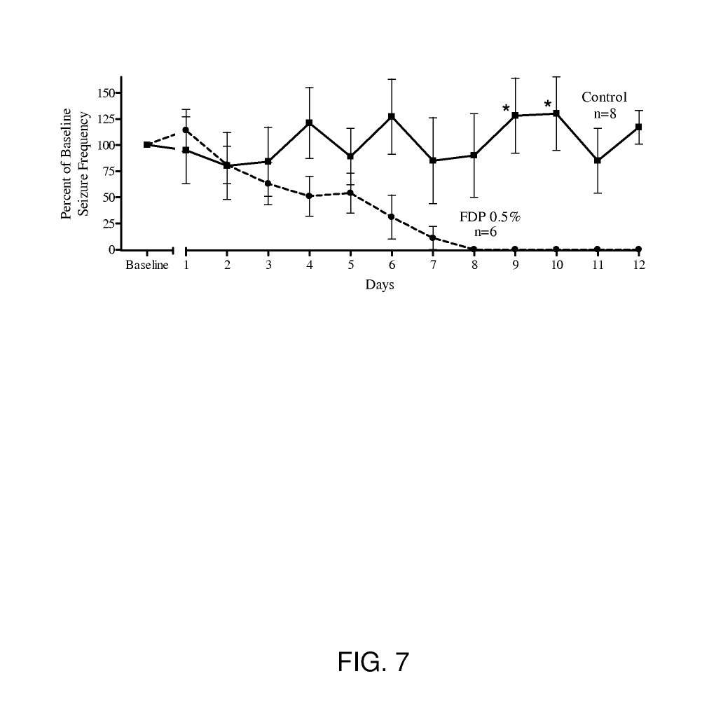

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

### I. General and Specific Embodiments of the Invention

The present invention generally concerns preventing epileptic seizure in a mammal, including a human, dog, cat, horse, pig, sheep, goat, and so forth. In particular cases, the epileptic seizure is prevented following delivery of fructose-1,6-bisphosphate to the individual. In the present invention, the anticonvulsant activity of F1,6BP was determined in three exemplary rat models of acute seizures. The efficacy of F1,6BP was compared to the efficacy of 2-deoxyglucose (an inhibitor of glucose uptake and glycolysis), the ketogenic diet, which decreases glycolysis by forcing the body to use fat instead of glucose, and valproate (VPA, a commonly prescribed anticonvulsant drug). In some embodiments, FDP is taken up and utilized by the brain. In specific embodiments, peripheral administration of FDP is altering metabolism within the brain without actually crossing the blood brain barrier. Therefore, in some embodiments direct measurements of FDP levels in the brain after peripheral administration are taken to characterize the effect of exogenously administered FDP on cerebral function.

Fructose-1,6-bisphosphate (F1,6BP) shifts the metabolism of glucose from glycolysis to the pentose phosphate pathway, and in some embodiments this provides anticonvulsant activity. In exemplary studies provided herein, the anticonvulsant activity of F1,6BP was determined in rat models of acute seizures induced by pilocarpine, kainic acid, or pentylenetetrazole, for example. The efficacy of F1,6BP was compared to that of 2-deoxyglucose (2-DG, an inhibitor of glucose uptake and glycolysis), valproic acid (VPA) and the ketogenic diet. One hour before each convulsant, Sprague-Dawley rats received either saline (as seizure controls), F1,6BP (0.25, 0.5 or 1 g/kg), 2-DG (0.25 or 0.5 g/kg), or VPA (0.3 g/kg). Additional animals received the ketogenic diet (starting at 20 or 60 days old). Time to seizure onset, seizure duration, and seizure score were measured in each group. F1,6BP had dose-dependent anticonvulsant activity in all three models, while VPA had partial efficacy. 2-DG was only effective in the pilocarpine model. The ketogenic diet had no effect in these models. F1,6BP was also partially effective when given at the first behavioral seizure after pilocarpine. Administration of sodium lactate, which bypasses the block in the glycolytic pathway, abolished the anticonvulsant activity of 2-DG in the pilocarpine model, but only decreased the efficacy of F1,6BP. These data demonstrate that F1,6BP has significant anticonvulsant efficacy.

Furthermore, exogenously administered fructose-1,6-diphosphate (FDP) has been studied for its ability to protect tissue during hypoxia or ischemia. Recently, a clear effect of FDP on the central nervous system has raised the question whether FDP can get into the brain. In the present invention, FDP levels were measured in blood, brain, liver, kidney, muscle and fat after intraperitoneal administration of a single 0.5 g kg-1 dose of FDP to adult male Sprague-Dawley rats. A complete time course of the levels in blood and brain was determined. The levels of FDP in the blood and brain increase simultaneously, i.e. there is no lag in the increase in the brain. The levels of FDP fall to baseline in liver, kidney, muscle and fat by 12 hours, but remain elevated in blood and brain. However, levels in the blood at 12 hours are significantly decreased from the peak levels, while those in brain are not different from the peak levels, indicating that the kinetics of FDP in blood and brain are quite different. Stripping the endothelial cells from the brain tissue sample did not change the levels of FDP, indicating that FDP is not trapped in the capillary cells. Incubation of brain slices in a solution of FDP, followed by washing, raised tissue levels of FDP indication that FDP is taken up into cells within the brain. Finally, the studies demonstrate a significant increase in brain levels of FDP after oral administration. These data indicate that an oral formulation of FDP is useful for treatment of neurological disease. Although in particular embodiments the present invention concerns seizures from epilepsy, in alternative embodiments the present invention is useful for any seizure not related to epilepsy.

Fructose-1,6-bisphosphate may be obtained commercially, for example from Sigma-Aldrich Co. (St. Louis, Mo.).

### II. Epilepsy

Epilepsy, which may also be referred to as a seizure disorder, is a medical condition in an individual that comprises seizures affecting a variety of functions, both mental and physical. A seizure occurs upon malfunction of the electrical system of the brain, wherein brain cells keep firing instead of discharging electrical energy in a controlled manner. In some cases, this results in a surge of energy through the brain, producing unconsciousness and massive contractions of the muscles. In other cases, where only part of the brain is affected, the seizure may affect awareness, block normal communication, and produce a variety of undirected, uncontrolled, unorganized movements. Although the majority of seizures last about a minute or two, confusion may linger.

Epilepsy can be diagnosed with a variety of means, and often a combination of methods are utilized to provide a definitive diagnosis. For example, electroencephalography (EEG) records can detect abnormalities in the brain's electrical activity by measuring brain waves detected by electrodes placed on the scalp. Epileptics often have an abnormal pattern of brain waves, even during the absence of a seizure. However, although an EEG can be very useful in diagnosing epilepsy, it is not foolproof and may be corroborated by additional tests. In some situations, doctors may employ an experimental diagnostic technique that detects signals from deeper in the brain than an EEG referred to as a magnetoencephalogram (MEG). The MEG detects magnetic signals produced by neurons to permit monitoring of brain activity at different locations in the brain over time, revealing different brain functions. Another way to diagnose epilepsy is through brain scans, such as CT (computed tomography), PET (positron emission tomography), or MRI (magnetic resonance imaging). CT and MRI scans illustrate brain structure, whereas PET and an adapted kind of MRI called functional MRI (fMRI) are utilized to monitor the brain's activity and detect abnormalities in how it functions. SPECT (single photon emission computed tomography) is a type of brain scan that may be used to locate seizure foci in the brain. Finally, magnetic resonance spectroscopy (MRS) can identify dysfunctioning brain biochemical processes.

Seizures can be classified in many different types, and people may experience just one type of seizure or multiple types of seizures. The seizure type a person experiences depends upon the location and extent of the brain that is affected by the electrical disturbance that produces seizures. Experts classify seizures as generalized seizures (absence, atonic, tonic-clonic, myoclonic), partial (simple and complex) seizures, nonepileptic seizures and status epilepticus. Seizures can last from a few seconds to a few minutes. They can have many symptoms, from convulsions and loss of consciousness to some that are not always recognized as seizures by the person experiencing them or by health care professionals: blank staring, lip smacking, or jerking movements of arms and legs. Epileptic seizures may be triggered by a number of events, although in some cases the seizure results following failure to take proper medication, ingesting substances, hormone fluctuations, stress, sleep patterns and photosensitivity, for example.

Causes of epilepsy are often unknown, although in some cases the cause can include anything that can make a difference in the way the brain functions, for example head injury, lack of oxygen to the brain, brain tumor, genetic conditions (such as tuberous sclerosis), lead poisoning, problems in development of the brain before birth, and infections (meningitis or encephalitis, for example).

If an underlying correctable brain condition is the cause of epilepsy, surgery may stop seizures. Seizure-preventing medications (such as carbamazepine, Carbatrol®, Clobazam, Clonazepam, Depakene®, Depakote®, Depakote ER®, Diastat, Dilantin®, Felbatol®, Frisium, Gabapentin®, Gabitril®, Inovelon®, Keppra®, Klonopin, Lamictal®, Lyrica, Mysoline®, Neurontin®, Oxcarbazepine, Phenobarbital, Phenylek®, Phenyloin, Rufinamide, Sabril, Tegretol®, Tegretol XR®, Topamax®, Trileptal®, Valproic Acid, Zarontin®, Zonegran, and Zonisamide, for example), a special ketogenic diet, complementary therapy or vagus nerve stimulation (VNS) may be employed to prevent seizure in addition to the methods and compositions of the present invention.

### III. Pharmaceutical Preparations

Pharmaceutical compositions of the present invention comprise an effective amount of fructose-1,6-bisphosphate, and in some cases, an additional agent, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains fructose-1,6-bisphosphate and, in some cases, an additional active ingredient, will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The composition of fructose-1,6-bisphosphate may comprise different types of carriers depending on whether it is to be administered in solid, liquid or aerosol form, and whether it need to be sterile for such routes of administration as injection. The present invention can be administered orally, although in alternative embodiments it is administered alintravenously, intradermally, transdermally, intrathecally, intraarterially, intraperitoneally, intranasally, intravaginally, intrarectally, topically, intramuscularly, subcutaneously, mucosally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The composition of fructose-1,6-bisphosphate may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include fructose-1,6-bisphosphate, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the fructose-1,6-bisphosphate may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the active compound may comprise between about 2% to about 75% of the weight of the unit, or between about 25% to about 60%, or between about 10% and 90%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, from about 5 mg/kg/body weight to about 100 mg/kg/body weight, etc., can be administered, based on the numbers described above.

A. Alimentary Compositions and Formulations

In preferred embodiments of the present invention, the fructose-1,6-bisphosphate is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In alternative embodiments, fructose-1,6-bisphosphate may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other preferred embodiments of the invention, the active compound fructose-1,6-bisphosphate may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Kits of the Invention

Any of the compositions described herein may be comprised in a kit. In a non-limiting example, fructose-1,6-bisphosphate, and in some cases an additional agent, is comprised in a kit. The kits will thus comprise any agent of the invention in suitable container means. In particular embodiments, the kits comprise a suitably aliquoted fructose-1,6-bisphosphate composition of the present invention. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional component(s) may be separately placed. However, various combinations of components may be comprised in a vial. The kits of the present invention also will typically include a means for containing the fructose-1,6-bisphosphate composition and any other reagent containers in close confinement for commercial sale. Such containers may include injection or blow molded plastic containers into which the desired vials are retained.

Irrespective of the number and/or type of containers, the kits of the invention may also comprise, and/or be packaged with, an instrument for assisting with the administration and/or placement of the ultimate composition within the body of an animal. Such an instrument may be a cup, syringe, pipette, forceps, and/or any such medically approved delivery vehicle.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods for Examples 2-4

All animal experiments were carried out in accordance with the National Institutes of Health guide for the care and use of laboratory animals (NIH publication 8023, revised 1996) and with the approval of the local Animal Use Committee. Unless indicated, all chemicals were obtained from Sigma Chemical Co (St. Louis Mo.). Male, Sprague Dawley rats weighing 50-74 g (young) or 147-309 g (adult) were used in this study. Acute seizures were induced by kainic acid (KA; Ocean Produce Int., Canada), pilocarpine or pentylenetetrazole (PTZ) as previously described (Lian et al 2006). These models were chosen because they initiate seizures by different mechanisms and the seizures have a relatively gradual onset compared to seizures initiated by stimulation. There was no difference in the body weight of the animals receiving the different convulsants. For pilocarpine, kainic acid and PTZ, the mean weights were 219±5 g (range 163-275 g), 215±7 g (range 183-258 g), and 216±13 g (range 147-309 g), respectively. The animals in the drug treatment groups were not different in weight from the animals in the control groups.

After administration of KA (10 mg/kg, ip), pilocarpine (scopolamine methyl bromide 1 mg/kg, s.c followed 15 min later by 300 mg/kg, ip pilocarpine) or PTZ (50 mg/kg, ip) (Lian et al., 2006), animals were continuously monitored for seizure activity for at least 5 h after KA or pilocarpine and for 30 min after PTZ. Behavioral seizures were scored by an investigator blinded to the treatment. Latency to the first wet dog shake after KA, latency to the first forelimb clonus (after pilocarpine, kainic acid or PTZ) and the score and duration of seizures were measured. When at least 1 hour had passed without any head bobbing (for pilocarpine) or any wet dog shakes (for KA), seizures were considered over. Status epilepticus lasting longer than 4 hours (for KA) or 5 hours (for pilocarpine) were assigned 4 and 5 hours for seizure duration. For animals receiving PTZ, the seizure duration was defined as the period of tonic-clonic seizures. EEG recording in the hippocampus was conducted as previously described (Lian et al., 2004). After anesthesia, a recording electrode was placed in a burr hole centered at 3.0 mm posterior to bregma, 1.8 mm lateral to the midline and then lowered 3.0 mm. A ground screw and wire was placed over the frontal region in another burr hole. This assembly was fixed to the skull with dental cement.

Each animal was assigned the score of the most severe seizure observed. The behavioral seizures induced by KA or pilocarpine were scored according to an adjusted version of the scale of Racine (Bough et al., 2002): stage 1, wet dog shakes after KA or trembling after pilocarpine; stage 2, head bobbing and stereotypes; stage 3, unilateral forelimb clonus; stage 4, bilateral forelimb clonus; stage 5, rearing and falling; stage 6, jumping and/or running followed by falling. Death within 24 h was assigned stage 7.

Adult rats received either D-F1,6BP, sodium valproate (VPA, 0.3 g/kg), 2-DG (0.25, 0.5 g/kg) or normal saline (vehicle) intraperitoneally followed 1 h later by one of the convulsants. The dose for VPA was based on experimental evidence that doses from 0.1 to 0.4 g/kg (ip) are effective in animal models (Bough and Eagles, 2001; Manent et al., 2007). Three doses of F1,6BP (0.25, 0.5 and 1 g/kg) were tested with this dosing schedule. Two additional groups were administered F1,6BP (0.5 or 1 g/kg, n=5) after pilocarpine. In this experiment, the F1,6BP was given intraperitoneally at the first behavioral seizure, which was chewing movements of the jaw. Two additional sets of animals were fed the classic ketogenic diet (No. F3666; Bio-Serv, Frenchtown, N.J.). One set of animals was given the diet beginning on postnatal day 22-26 (KD-Yng) and maintained on this diet for 4 weeks. Thus the seizures were tested in this group when the animals had reached approximately the same age (50-54 days) as the majority of the animals tested. The other set of animals started the diet as adults and remained on the diet for 10 days (KD-Adult). β-hydroxybutyrate levels (Clinical Pathology Laboratory, Texas Children's Hospital) were confirmed to be elevated to levels previously reported (Bough et al., 1999) using an additional 3 animals in each diet group (control, 0.11-0.31 mmol/l; KD-Yng, 0.72-0.87 mmol/l; KD-Adult, 1.5-2.7 mmol/kg).

The latency to seizure onset, seizure score and seizure duration were averaged across animals in each group. Comparisons between groups were done with an analysis of variance with Bonferroni post hoc test. Statistical difference was defined as p<0.05.

### Example 2

### Anticonvulsant Activity Of F1,6BP, 2-DG, Valproate and the Ketogenic Diet in the Pilocarpine Model

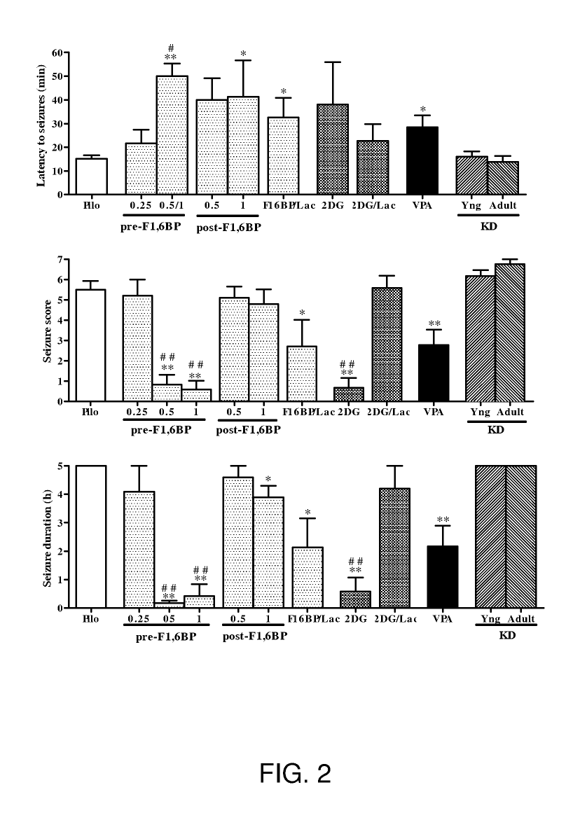

To begin to test the anticonvulsant activity of F1,6BP, pilocarpine, a cholinergic agonist, was used to produce the gradual onset of generalized seizures. All animals (n=10) pretreated with saline followed by pilocarpine had generalized seizures lasting at least 5 hours. The mean seizure score was 5.5±0.4 (FIG. 2A). The mean latency to forelimb clonus was 14±1 min. In addition, four out of ten animals died within 24 h.

Pretreatment with F1,6BP had a dose-dependent anticonvulsant effect. The lowest dose (0.25 g/kg, n=5) had no effect on the seizure parameters. In animals pretreated with 0.5 g/kg of F1,6BP, only 3 out of 10 animals had a seizure score ≧3. In animals pretreated with 1 g/kg, 2 out of 10 had a seizure score ≧3. In these 5 animals, the latency to the seizures was significantly increased. In animals pretreated with 0.5 or 1 g/kg of F1,6BP, seizure duration and seizure score were significantly decreased. To identify electrographic seizures that do not have a behavioral component, hippocampal EEG recordings were conducted in 2 saline-pretreated rats and 4 rats pretreated with 1 g/kg F1,6BP followed by pilocarpine. The four rats treated with F1,6BP had no behavioral or electrographic seizures (FIG. 2B). To determine whether F1,6BP could alter the course of the pilocarpine-induced seizures once they had begun, either 0.5 or 1 g/kg (n=5 for each dose) was administered at the very first sign of seizure activity, which was chewing movements. The higher dose (1 g/kg) significantly slowed the progression of the seizures as measured by an increase in the latency to forelimb clonus and decrease in seizure duration.

Pretreatment with 2-DG was also effective against pilocarpine-induced seizures; decreasing seizure duration and seizure score. After treatment with 2-DG at 0.25 g/kg (n=6), only one animal had a stage 3 seizure. Pretreatment with VPA (0.3 g/kg, ip, n=9) significantly reduced the mean seizure score and duration, but not to the extent of F1,6BP and 2-DG. In these animals, 6 out of 9 had forelimb clonus and one died. The ketogenic diet had no effect on any measured seizure parameters. In the group that received the ketogenic diet for 4 weeks (KD-Yng, n=6), 2 out of 6 died within 24 h. Those that received the ketogenic diet for 10 days as adults (n=4) all had severe clonus and three died within 24 h.

### Example 3

### Effect of Exogenous Lactate on the Anticonvulsant Efficacy of F1,6BP and 2-DG

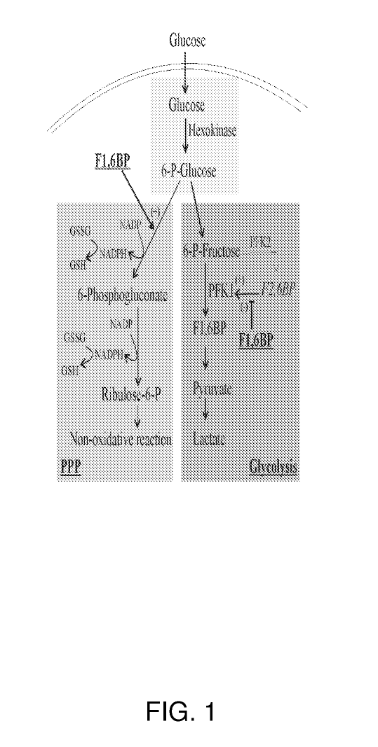

F1,6BP and 2-DG both reduce metabolism of glucose through the glycolytic pathway, but F1,6BP also increases the flux of glucose through the pentose phosphate pathway. This increase may contribute to the anticonvulsant efficacy of F1,6BP. To investigate this, exogenous sodium lactate (0.5 g/kg, ip) was administered 30 minutes after F1,6BP (1 g/kg, ip) or 2-DG (0.25 g/kg, ip). Thirty minutes later, the animals received pilocarpine (300 mg/kg, ip). Lactate should provide a substrate for the glycolytic pathway beyond the point of inhibition by either F1,6BP or 2-DG (FIG. 1).

In animals pretreated with F1,6BP and lactate, 3 of 7 had ≧stage 3 seizures and an increase in latency to seizures (FIG. 2A). The seizure score and seizure duration were significantly decreased compared to the seizure control group. In animals pretreated with 2-DG and lactate, severe seizures (stage 4-5) were noted in all animals (n=5). The seizure score and duration were not different from those in the seizure control group. These data demonstrate that lactate abolishes the anticonvulsant action of 2-DG, but only reduces the efficacy of F1,6BP.

### Example 4

### Anticonvulsant Activity of F1,6BP, 2-DG, Valproate and the Ketogenic Diet in the Kainic Acid Model

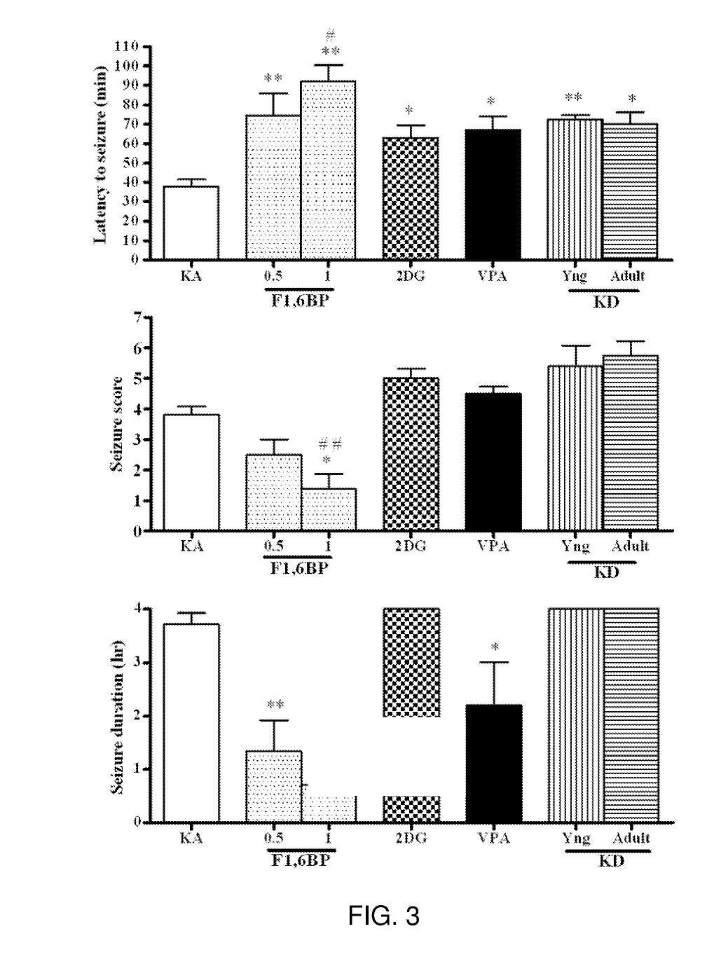

To determine whether F1,6BP is effective in other models, additional animals were given kainic acid, a glutamate receptor agonist, to induce partial seizures with secondary generalization. In animals pretreated with saline followed by KA (n=10), one had only wet dog shakes (stage 1, FIG. 3) and the remainder had severe seizures (≧stage 3). The latency to the first wet dog shake was 38±4 min, the latency to the first forelimb clonus was 58±2 min, values for seizure score and duration were 3.7±0.3 and 3.7±0.2 h, respectively. No animals died. F1,6BP had a dose-dependent effect on the seizures. At 0.25 g/kg (n=6), F1,6BP had no effect. At 0.5 or 1 g/kg, F1,6BP significantly delayed the onset of seizures, and decreased the seizure score and seizure duration. Two out of 8 animals pretreated with 0.5 g/kg had no seizures, three had mild seizures (wet dog shakes or head bobbing) and the remaining 3 had severe seizures (mean seizure score 2.5±0.5). Three out of 8 pretreated with 1 g/kg had no seizures, 3 had mild seizures (wet dog shakes or head bobbing), and 2 had severe seizures, for an average seizure score of 1.4±0.5 for the entire group. The mean latency to first forelimb clonus in this group was 105±7 min, which was statistically difference from the control group.

2-DG, at 0.25 g/kg (n=6), delayed the appearance of the first wet dog shake, but not the first forelimb clonus and had no effect on the other parameters. At 0.5 g/kg, 2-DG had no additional activity (n=3, data not shown). Although animals pretreated with VPA (0.3 g/kg, ip, n=6) had severe seizures with a seizure score of 4.5±0.2, VPA significantly delayed the appearance of wet dog shakes, but not the appearance of forelimb clonus and decreased the duration of seizures. The ketogenic diet only delayed the appearance of wet dog shakes, but not the appearance of forelimb clonus. All animals treated with the ketogenic diet (n=5, KD-Yng and n=4, KD-Adult) had severe seizures and three died within 24 h.

### Example 5

### Anticonvulsant Activity of F1,6BP, 2-DG, Valproate and the Ketogenic Diet in the PTZ Model

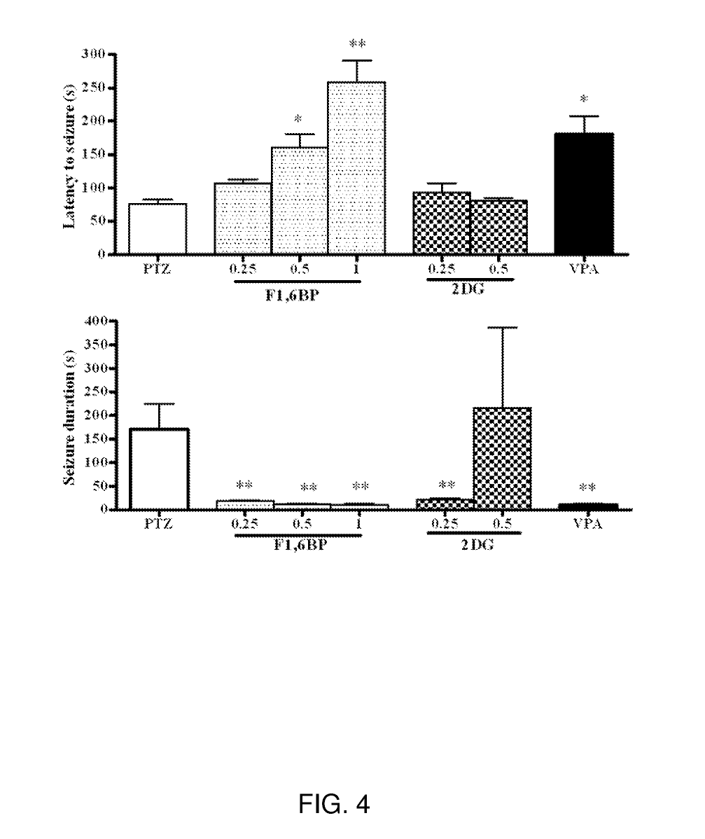

To further test the anticonvulsant activity of F1,6BP, PTZ, a GABA antagonist, was used to induce a single generalized seizure. All rats (n=9) pretreated with saline had generalized tonic-clonic seizures after PTZ (FIG. 4). The latency to generalized tonic-clonic seizures was 76±6 sec and the duration of the seizures was 170±54 sec. F1,6BP had a dose-dependent effect on the latency to the seizures (0.25 g/kg, n=6, latency 107±6 sec; 0.5 g/kg, n=6, latency 161±16 sec; 1 g/kg, n=7, latency 259±32 sec). All doses reduced the seizure duration to the same degree (0.25 g/kg, n=6, duration 18±2 sec; 0.5 g/kg, n=6, duration 12±1 sec; 1 g/kg, n=7, duration 11±3 sec). Three out of 8 animals who received 1 g/kg of F1,6BP had no seizures.

All animals that received 2-DG had seizures and the seizure latency was not increased (n=5 for both 0.25 and 0.5 g/kg). The seizures were significantly shortened by the 0.25 g/kg dose. In animals pretreated with VPA (0.3 g/kg, n=8), two animals had no generalized tonic-clonic seizures, and six had a significantly longer seizure latency (185±27 sec). VPA also significantly decreased the seizure duration (9±4 sec).

### Example 6

### Exemplary Materials and Methods for Example 7

Adult male Sprague-Dawley rats (170-220 g) were used to determine the kinetics of fructose-1,6-diphosphate (FDP). Levels of FDP were determined in blood and tissue samples as previously described (Gerhard, Methods of Enzymatic Analysis, Vol VI, Ed. Bergmeyer, H U, Academic Press NY, pp. 342-350). All chemicals and enzymes for the assay were obtained from Sigma Chemical Co (St. Louis Mo.). Animals were administered a single dose of 0.5 g kg-1 FDP (250 mg ml-1 dissolved in 0.1M phosphate buffered saline, pH 7). In vivo studies utilized the dicalcium salt of FDP, which has a purity of ˜70%. Preliminary studies demonstrated no difference in efficacy between the trisodium salt, with a purity of >98% and the dicalcium salt. At the designated time, animals were anesthetized with 1 g kg-1 urethane. After deep anesthesia, whole blood was obtained from a cardiac puncture. One ml of whole blood was added to 5 ml of perchloric acid (0.6 ml l-1) and mixed. After centrifugation at 3,500 rev min-1 for 10 min at 4° C., the supernatant was removed and set aside. The sediment was re-suspended in 1 ml of the perchloric acid solution and 1 ml of distilled water and centrifuged. The resulting supernatant was combined with the first one and the pH was adjusted to 3.5 with potassium carbonate (5 mol l-1). The final volume was brought to 7 ml and the solution was allowed to sit in ice for 15 min. The supernatant was used for determination of FDP levels.

Immediately after removal of the whole blood sample, the rat was perfused through the heart with ice-cold 0.01M phosphate buffered saline. Tissue samples from liver, kidney, skeletal muscle (from thigh) and intra-abdominal fat were obtained and then the brain was removed and dissected into hippocampus, cerebral cortex, cerebellum and rest of brain. In initial experiments, the different brain regions did not give statistically different results, so the values for these 4 samples were averaged to give a mean FDP level in the brain for each animal. All tissue samples were weighed and then homogenized in 5 ml of ice-cold perchloric acid (0.6 mol l-1) as quickly as possible. The homogenates were then treated was described above for whole blood.

Actual levels of FDP were determined by monitoring absorbance of reduced nicotinamide adenine dinucleotide (NADH) and treating the sample with aldolase (EC 4.1.2.13, 45 units mg-1 diluted 1:27 with distilled water), the enzyme which cleaves FDP into dihydroxyacetone phosphate (DAP) and D-glyceraldehyde 3-phosphate (GAP). DAP and GAP are interconverted by the enzyme triosephosphate isomerase (TIM, EC 5.3.3.1, 5290 units mg-1 diluted 1:120 with distilled water). Glycerol-3-phosphate dehydrogenase (GDH, EC 1.1.1.8, 252 units mg-1 diluted 1:100 with distilled water) catalyzes the reduction of DAP by NADH. For the FDP measurements, 1.5 ml of tetraethyl ammonium buffer (TEA, 0.4 mol l-1, pH 7.6 with EDTA 40 mmol l-1) was added to 1 ml of the sample in a 4 ml cuvette. Then 0.1 ml of 5 mmol l-1 β-NADH, 0.4 ml of distilled water and 0.01 ml of the enzymes TIM and GDH were added and the cuvette inverted to mix the solutions. After 5 min the absorbance was read 3 times at 340 nm, each reading 3 min apart. The average of these readings is the initial absorbance (Ai). This step removes any DAP or GAP in the sample and determines the baseline absorbance. Aldolase (0.01 ml) was then added and mixed to cleave FDP into DAP and GAP. Nine minutes after addition of the aldolase, the final absorbance (Af) was determined by 3 readings at 340 nm, each 6 minutes apart. The concentration of FDP in the sample was proportional to the difference in the initial and final absorbance. Two moles of NADH are oxidized for each mole of FDP. Blanks and a FDP standard sample were run in parallel with every assay. Each blank had 1.6 ml of the TEA buffer, 1.4 ml water and the mixture of TIM and GDH. For the positive control, the sample was replaced with 1 ml of a solution containing 200 μg FDP per ml of phosphate buffered saline. The trisodium salt of FDP, with a purity of >98%, was utilized for all of the positive control samples and for the standard curves. Levels of FDP in blood were determined per ml, while levels in tissue samples were determined as a function of tissue weight.

In additional samples, a capillary depletion protocol was carried out to remove endothelial cells from the brain samples (Triguero et al., 1990). The cortex was harvested as described above and homogenized in 3 ml perchloric acid. Four milliliters of a 26% dextran (low fraction) solution was then added and the sample was homogenized again. The homogenates were then centrifuged at 5400 rev min-1 for 15 min at 4° C. The supernatant and pellet were carefully separated and processed separately for FDP levels as described above.

Additional experiments determined the uptake of FDP in brain slices in vitro. Male Sprague-Dawley rats (150-160 g, n=5) were anesthetized with a ketamine cocktail (mixture of ketamine (42.8 mg ml-1), xylazine (8.6 mg ml-1), acepromazine (1.4 mg ml-1); dose 0.5-0.7 ml kg-1) and then perfused through the heart with an ice-cold solution containing 110 mM choline Cl, 2.5 mM KCl, 1.25 mM NaH₂PO₄, 25 mM NaHCO₃, 10 mM glucose, 0.5 mM CaCl₂, and 7.5 mM MgCl₂ and oxygenated with 95% O2/5% CO₂. The brain was rapidly removed and cut transversely along the septo-temporal axis. Both halves of the brain were cut into 6-8 sagittal sections, 400 μm thick on a Vibratome (Technical Products, St. Louis, Mo.). The slices were incubated at 32° C. for at least 30 min in an artificial cerebrospinal (ACSF) solution containing 125 mM NaCl, 2.5 mM KCl, 1.25 mM NaH₂PO₄, 25 mM NaHCO₃, 10 mM glucose, 2 mM CaCl₂, 2 mM MgCl₂, 1.3 mM ascorbate and 3 mM pyruvate, equilibrated with 95% O₂/5% CO₂. Half of the slices were then transferred to a container with ACSF plus 500 μg ml-1 FDP (˜1 mM). The other half of the slices remained in ACSF. After 1 hour of incubation, all slices were washed 3 times for 15 min each in ice-cold ACSF. Immediately after washing, both control and FDP-treated slices were weighed and then homogenized in 5 ml ice-cold perchloric acid. Determination of FDP levels was carried out as described above.

The time course for FDP in blood and brain was analyzed with a 2-way ANOVA comparing to control levels as a function of time. Other comparisons were done with a 1-way ANOVA or grouped t-test as appropriate. Significance was set at p<0.05.

### Example 7

### Pharmacokinetics of Fructose-1,6-Diphosphate after Intraperitoneal and Oral Administration to Adult Rats

Initial results with the FDP assay demonstrated that the changes in absorbance were linear between up to at least 45 μg ml-1, which is in the range of values obtained in both blood and tissue. In addition, when 1 ml of 200 μg ml-1 of fructose was added in place of the sample in the assay (for a final concentration of 66.7 μg ml-1), there was no change in the absorbance. This indicates that the assay is specific for the phosphorylated form of fructose. A sample blank and FDP-positive control (final concentration of 33.5 μg ml-1) were included in every assay run. The results for naïve animals in blood are within the range previously reported for humans at baseline (1.13 mg dl-1) and after administration of FDP (3.39 mg dl-1, Markov et al., 2000). The control group includes both vehicle control (n=3) and naïve (n=9) animals. There was no difference in these groups in any tissue, so the results have been pooled.

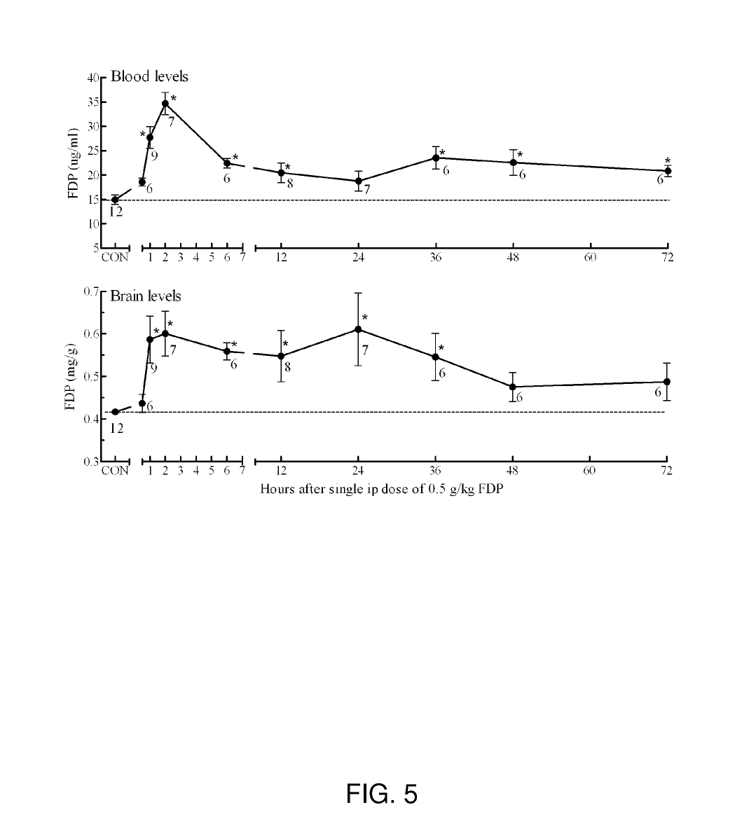

Adult male Sprague-Dawley rats were administered a single dose of 0.5 g kg-1 FDP (250 mg ml-1 dissolved in 0.01M phosphate buffered saline, pH 7) and sacrificed at various times (FIG. 5). This dose and route of administration has previously been shown to be an effective anticonvulsant (Lian et al., 2007). There was a relatively rapid rise in FDP levels in blood. An increase was seen as early as 30 minutes after administration and there was a significant increase at 1 hour. The levels peaked at 2 hours after administration and by 6 hours had fallen more than halfway back to baseline levels. The levels then remained elevated out to 72 hours after a single dose. The level of FDP in the blood at 12 hours after administration was significantly elevated compared to control values, but also significantly decreased compared to the peak levels at 2 hours. The levels in the brain also rose very promptly, peaking 1-2 hours after administration of FDP. The levels then fell slightly, but remained significantly elevated at 36 hours. The level of FDP in the brain at 12 hours was not significantly different than the levels at 1 and 2 hours after administration of the FDP indicating a sustained elevation of FDP in the brain. Thus the levels in the blood and brain do not follow the same kinetic profile.

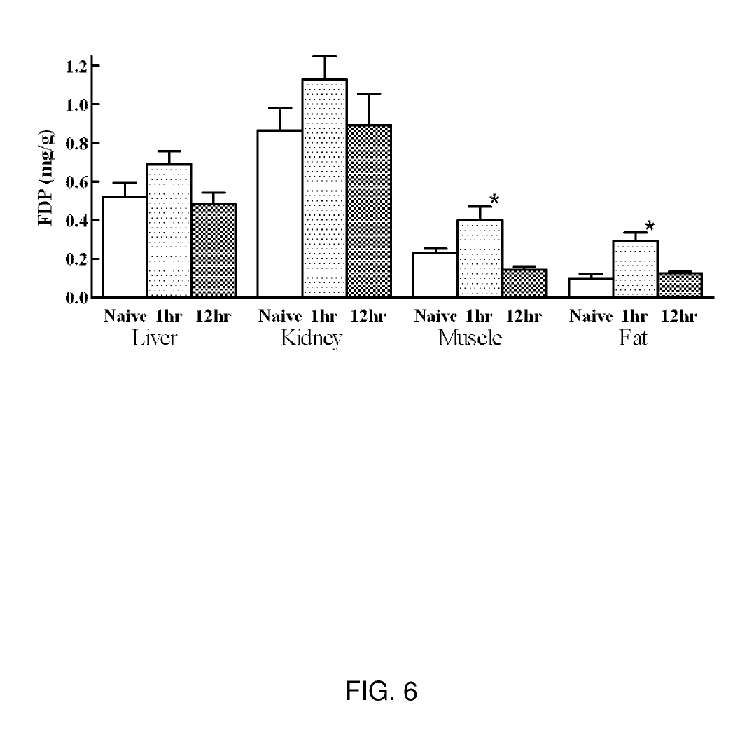

To test the handling of FDP in other tissues, animals were perfused with ice cold phosphate buffered saline to remove blood from the tissues of interest. The levels of FDP in liver, kidney, skeletal muscle and fat were determined in naïve animals and 1 and 12 hours after administration of 0.5 g kg-1 FDP (n=6, FIG. 6). Based on the data from blood and brain, the 1 hour time point is expected to have the peak levels of FDP. The 12 hour time point was chosen because at this time the levels in the blood have returned closer to baseline, while the levels in the brain are still elevated. Baseline levels in the different tissues are quite different, but the increase in FDP at 1 hour appears to be roughly equivalent in all tissues measured. There was an increase of around 0.2 mg g-1 (increase of 0.17 mg g-1 (muscle and liver) to 0.26 (kidney)). The increase at 1 hour only reached statistical significance in muscle and fat, most likely due to the low baseline levels in these tissues. In liver, kidney, muscle and fat, the levels of FDP had returned to near baseline at 12 hours indicating that the levels in these tissues mirror more closely the levels in blood than the levels of FDP in brain tissue.

To determine whether the FDP was getting into the brain tissue, two additional experiments were carried out. First, the endothelial cells were stripped from the brain samples. The ratio of FDP in the stripped cortex to the FDP in whole cortex was 0.86±0.04 in control animals (n=3). In animals treated with FDP the ratio was 0.91±0.01 (n=7), which is not statistically different from the control animals (grouped t-test). Within the group of animals treated with FDP, 4 were sacrificed ≦2 hours after treatment and the remaining 3 were sacrificed more than 12 hours after treatment with FDP. There was no difference in the levels of FDP in the endothelial cells in any of these groups compared to control. These results show that FDP is not trapped in the endothelial cells, but does cross into the brain parenchyma. In the second experiment brain slices were prepared and incubated in ACSF containing 500 μg ml-1 FDP to determine whether FDP could be transported into cells within the brain tissue. After incubation, slices were washed to remove FDP from the extracellular space. Incubation for 1 hour in 500 μg ml-1 FDP resulted in a significant increase in tissue levels of FDP (0.22±0.013 mg g-1 compared to 0.14±0.004 mg g-1 in control slices, p=0.0006 with a grouped t-test).

Because FDP does appear to cross membranes into a variety of tissues in the body after intraperitoneal administration, we chose to determine whether FDP had oral bioavailability. FDP was added to water at a concentration of 0.5% (pH=7). Five animals received only this water to drink for 7 days. By measuring the amount of solution in the water bottle at the beginning and again at the end of the 7 day treatment period, it was estimated that an average of 226±13 ml (mean±SEM) was consumed. This translates into approximately 160 mg of FDP consumed per day. If there was significant water spillage or leakage, then the total FDP consumption would be less. Levels of FDP in the blood and brain of these animals was significantly increased compared to naïve animals. After oral administration, the levels in the blood were 22.2±3.3 μg ml-1 (mean±SEM, n=5, compared to 15.0±1.0 μg ml-1 in naïve animals, n=11) and in the brain were 0.50±0.024 mg g-1 (mean±SEM, n=5, compared to 0.42±0.01 mg g-1 in naïve animals, n=11), which are not significantly different from the levels in blood and brain 12 hours after a single intraperitoneal dose.

### Example 8

### Anticonvulsant Action of Oral Administration of Fructose-1,6-Bisphosphate

Animals (n=14) were treated with pilocarpine to induce status epilepticus and subsequent spontaneous seizures. Four weeks later daily observation was begun and the number of seizures per day was determined for at least 6 days. The mean number of seizures per day over a 6 day period was determined for each animal and this was normalized to 100%. Half of the animals then had FDP added to the drinking water at a concentration of 0.5%. The number of seizures on each day was determined for each animal and calculated as percent of the baseline. The mean (±SEM) across animals in each treatment group was then calculated and plotted as a function of the days of treatment. By day 7, only 1 animal in the FDP-treatment group had a single seizure. Overall, the data was analyzed with a 2-way ANOVA comparing drug treatment against time and baseline rate of seizures. The FDP treated group was significantly different than the non-treatment (control) group. Post-hoc analysis determined that specifically on day 9 and 10 (*) the control was different than FDP-treated. Thus, oral administration of FDP has anticonvulsant activity against the generalized tonic-clonic seizures that are observed after pilocarpine-induced status epilepticus.

### Example 9

### Significance of the Present Invention

This invention demonstrates that F1,6BP, a regulator of glucose utilization by inhibition of glycolysis and enhancement of metabolic flux through the pentose phosphate pathway, has anticonvulsant efficacy against acute seizures triggered by exemplary compositions, including a cholinergic agonist (pilocarpine), a glutamate receptor agonist (kainic acid) and a GABA antagonist (PTZ). F1,6BP was also able to significantly modify the pilocarpine-induced seizures when administered after the seizures had begun. 2-DG (an inhibitor of glycolysis) had some efficacy in these models, but was not as consistently effective as F1,6BP. The ketogenic diet had limited efficacy. The data with the ketogenic diet are consistent with that in the literature for activity against acute seizures induced by kainic acid (Bough et al., 2002; Noh et al., 2003), PTZ or other animal models of seizures (Nylen et al., 2005). The finding that F1,BP is effective in all models tested may be due to its action on a final common pathway in epileptogenesis that is independent of the mechanism of seizure initiation.

In some embodiments, the reduction in glycolysis (metabolism of glucose to pyruvate) is responsible for the anticonvulsant action of F1,6BP. The ketogenic diet, which forces the body to use fat instead of carbohydrates, has been used to manage refractory epilepsy in children (Freeman et al., 2007). Recently, a decrease in glycolysis has been suggested to be the mechanism of this diet (Greene et al., 2001; Greene et al., 2003). 2-deoxyglucose (2-DG) was recently reported to have anticonvulsant activity (Garriga-Canut et al., 2006). 2-DG blocks glucose uptake and also inhibits glycolysis by inhibiting hexokinase, the enzyme that phosphorylates glucose (Bissonnette et al., 1996). In the present invention, exogenous lactate was given to provide substrate for cells beyond the point of inhibition in the glycolytic pathway (FIG. 1). The anticonvulsant action of 2-DG was completely reversed by lactate, indicating that in some embodiments the inhibition of glycolysis underlies its anticonvulsant action. The effect of F1,6BP was only partially reversed. This is presumably related to the ability of F1,6BP to increase flux of glucose into the pentose phosphate pathway (Kelleher et al., 1995; Espanol et al., 1998) and increase levels of GSH (Vexler et al., 2003), a potent endogenous anticonvulsant (Abe et al., 2000). Addition of lactate would not alter this action of F1,6BP. Together these data indicate that decreasing glycolysis pharmacologically is an effective anticonvulsant mechanism.

F1,6BP has been administered to humans with no reported toxicity. It has been safely used in patients with myocardial damage (Munger et al., 1994), ischemic heart disease (Pasotti et al., 1989; Liu et al., 1998), ischemic stroke (Karaca et al., 2002) and during coronary artery bypass graft surgery (Riedel et al., 2004). It has also been found to be safe in trials with healthy volunteers in doses from 5 to 15 g (Ripari et al., 1988; Markov et al., 2000). However, intravenous administration of F1,6BP has been shown to have an LD50 in rats of 1,068 mg/kg (Nunes et al., 2003). Although there are no reports of F1,6BP testing in humans with epilepsy, clinical testing may be performed by standard methods in the art.

If F1,6BP, the ketogenic diet and 2-DG are all altering seizure susceptibility by an action on glycolysis, then in some embodiments F1,6BP has fewer side effects. It has been hypothesized that the efficacy of the ketogenic diet is due to the reduction in glucose availability (Greene et al., 2003) and 2-DG inhibits glucose uptake (Bissonnette et al., 1996). Therefore, both of these treatments would result in an overall decrease in glucose utilization (including through the pentose phosphate pathway). F1,6BP shifts metabolism of glucose from the glycolytic pathway to the pentose phosphate pathway in astrocytes (Kelleher et al., 1995). This provides two apparently beneficial effects—reducing glycolysis and increasing glutathione production. A decrease in overall glucose utilization by the ketogenic diet may impair cognitive function (Zhao et al., 2004). Additionally, subcutaneous administration of 0.3 μmol 2-DG to a chick has been shown to inhibit memory consolidation (Gibbs and Summers, 2002). Because F1,6BP allows glucose utilization, in specific embodiments it results in less cognitive impairment making it suitable for clinical use as an anticonvulsant.

The studies provided herein demonstrate that peripheral administration of FDP raises levels in tissues throughout the body. The levels of FDP in the blood and brain increase simultaneously, i.e. there is no lag in the increase in the brain. The levels of FDP fall to baseline in liver, kidney, muscle and fat by 12 hours, but remain elevated in blood and brain. However, levels in the blood at 12 hours are significantly decreased from the peak levels, while those in brain are not different from the peak levels, suggesting that the kinetics of FDP in blood and brain are quite different. Further studies indicate that FDP is taken up into the cells in the brain and not trapped in the endothelial cells of the brain. Finally, the studies demonstrate a significant increase in brain levels of FDP after oral administration. These data demonstrate that exogenous administration of FDP results in a significant increase in levels of FDP in the brain and also indicate that an oral formulation of FDP is useful for treatment of neurological disease.

Studies have shown that FDP can cross lipid bilayers in a dose-dependent manner (Ehringer et al., 2000). It has also been hypothesized that FDP can cross cell membranes via either a band 3 or a dicarboxylate transporter. This was tested in isolated rat heart myocytes (Hardin et al., 2001) where it was concluded that since fumarate and malate could cross the plasma membrane that a dicarboxylate transport system is present on these cells. A band 3 inhibitor had no effect on production of [¹³C]lactate from [¹³C]FDP in these cells, but fumarate, which will compete for transport on the dicarboxylate transporter, did inhibit metabolism of [¹³C]FDP. The data provided herein is consistent with transport of FDP into cardiac myocytes by a dicarboxylate transport system. This invention also found no conversion of FDP to fructose. The sodium-dicarboxylate cotransporter family includes 2 proteins found in humans (SLC13A2 and SLC13A3, Markovich and Murer, 2004) that are reported to transport succinate, citrate and α-ketoglutarate. SLC13A3 is reported to be present in brain tissue. Searching the Allen Institute Brain Atlas, the mRNA for SLC13A3 appears to be in very low levels in the brain. In some sections, positive staining appears in choroids plexus and possibly ependymal cells. The expression levels for the mitochondrial dicarboxylate transporter (SLC25A10) show staining in a more uniform distribution throughout the brain in cell bodies. In the hippocampus, it appears that the mRNA for this transporter is found at moderate levels in principal neuronal cells only. Lower levels are seen in the cortex in layers II and IV/V. It does not appear to be expressed in interneurons or glial cells. Therefore, in some embodiments FDP crosses the blood brain barrier and is transported into cells via SLC25A10, and then metabolism in neurons is to be changed more than metabolism in glial cells. If FDP is moving through the body by diffusion through membranes, then both neuronal and glial metabolism are altered in a similar fashion, in particular embodiments. In some cases, more than one process may also be involved in the movement of FDP into and through the brain. Evidence in cardiac myoctyes indicates that at least 2 processes are involved in the entry of FDP (Wheeler et al., 2004).

It is noteworthy that the levels of FDP remain elevated in the brain long after they have fallen in the peripheral tissues. FDP is a normal cellular constituent and, as such, has a normal route of metabolism and cellular regulation. When exogenous FDP is administered, in some embodiments a cell would respond by increasing the metabolism of FDP, but this is not consistent with the data in provided herein. In an alternative embodiment, the exogenously administered FDP alters metabolism to the extent that more FDP is generated within the cells, maintaining the overall level. Again, while possible, this does not seem likely since there are no other examples of this type of interaction. The ability of various tissues to hydrolyze FDP has been measured in organ extracts and brain had the lowest level (Rigobello and Galzigna, 1982). Therefore, in some embodiments levels remain elevated because of decreased metabolism. However, if FDP can diffuse across membranes, then one would expect FDP to diffuse back out of the brain as the levels fall in the blood. The data indicate that the FDP is trapped in the brain. This supports a one-way transport system with limited tissue metabolism of FDP. Irrespective of the mechanism, the kinetics of FDP in the brain indicate that less frequent dosing would be needed to maintain therapeutic levels of FDP in the brain compared to other tissues. The data also indicate that oral dosing can result in a significant elevation of levels of FDP in the brain.

### REFERENCES

All patents and publications mentioned in the specification are indicative of the levels of those skilled in the art to which the invention pertains. All patents and publications are herein incorporated by reference in their entirety to the same extent as if each individual publication was specifically and individually indicated to be incorporated by reference.

### PATENTS AND PATENT APPLICATIONS

U.S. Pat. No. 5,399,363

U.S. Pat. No. 5,466,468

U.S. Pat. No. 5,543,158

U.S. Pat. No. 5,580,579

U.S. Pat. No. 5,629,001

U.S. Pat. No. 5,641,515

U.S. Pat. No. 5,792,451

U.S. Pat. No. 6,613,308

### PUBLICATIONS

Abe K, Nakanishi K, Saito H (2000). The possible role of endogenous glutathione as an anticonvulsant in mice. Brain Res 854:235-238.
Bergren S K, Chen S, Galecki A, Kearney J A. Genetic modifiers affecting severity of epilepsy caused by mutation of sodium channel Scn2a. Mamm Genome. 2005 September; 16(9):683-90.
Bissonnette P, Gagne H, Blais A, Berteloot A (1996). 2-Deoxyglucose transport and metabolism in Caco-2 cells. Am J Physiol 270:153-162.
Bough K J, Eagles D A (2001). Comparison of the anticonvulsant efficacies and neurotoxic effects of valproic acid, and the ketogenic diet. Epilepsia 42:1345-1353.
Bough K J, Gudi K, Han F T, Rathod A H, Eagles D A (2002). An anticonvulsant profile of the ketogenic diet in the rat. Epilepsy Res 50:313-325.
Bough K J, Valiyil R, Han F T, Eagles D A. (1999). Seizure resistance is dependent upon age and calorie restriction in rats fed a ketogenic diet. Epilepsy Res. 35:21-28.
Chugani H T, Chugani D C (1999). Basic mechanisms of childhood epilepsies: studies with positron emission tomography. Adv Neurol 79:883-891.
Ehringer W D, Niu W, Chiang B, Ou-Li W, Gordon L, Chien S. Membrane permeability of fructose-1,6-diphosphate in lipid vesicles and endothelial cells. Mol Cell Biochem. 2000; 210: 35-45.
Espanol M T, Litt L, Hasegawa K, Chang L H, Macdonald J M, Gregory G, James T L, Chan P H (1998). Fructose-1,6-bisphosphate preserves adenosine triphosphate but not intracellular pH during hypoxia in respiring neonatal rat brain slices. Anesthesiology 88:461-472.
Farias L A, Smith E E, Markov A K Prevention of ischemic-hypoxic brain injury and death in rabbits with fructose-1,6-diphosphate. Stroke. 1990; 21: 606-13.
Farias L A, Sun J, Markov A K. Improved brain metabolism with fructose 1-6 diphosphate during insulin-induced hypoglycemic coma, Am. J. Med. Sci. 1989; 297: 294-299.
Freeman J M, Kossoff E H, Hartman A L (2007). The ketogenic diet: one decade later. Pediatrics 119:535-543.
Garriga-Canut M, Schoenike B, Qazi R, Bergendahl K, Daley T J, Pfender R M, Morrison J F, Ockuly J, Stafstrom C, Sutula T, Roopra A (2006). 2-Deoxy-D-glucose reduces epilepsy progression by NRSF-CtBP-dependent metabolic regulation of chromatin structure. Nat Neurosci 9:1382-1387.
Gerhard M. D-Fructose 1,6 bisphosphate, dihydroxyacetone phosphate and D-glyceraldehyde 3-phosphate. Methods of Enzymatic Analysis, Vol VI, Ed. Bergmeyer, H U, Academic Press NY, pp. 342-350.
Gibbs M E, Summers R J (2002). Effects of glucose and 2-deoxyglucose on memory formation in the chick: interaction with beta(3)-adrenoceptor agonists. Neuroscience 114:69-79.
Greene A E, Todorova M T, McGowan R, Seyfried T N (2001). Caloric restriction inhibits seizure susceptibility in epileptic EL mice by reducing blood glucose. Epilepsia 42:1371-1378.
Greene A E, Todorova M T, Seyfried T N (2003). Perspectives on the metabolic management of epilepsy through dietary reduction of glucose and elevation of ketone bodies. J Neurochem 86:529-537.
Hardin C D, Lazzarino G, Tavazzi B, Di Pierro D, Roberts T M, Giardina B, Rovetto, M J. Myocardial metabolism of exogenous FDP is consistent with transport by a dicarboxylate transporter. Am J Physiol Heart Circ Physiol. 2001; 281: H2654-H2660.
Hardin C D, Roberts T M. Metabolism of exogenously applied fructose 1,6-bisphosphate in hypoxic vascular smooth muscle. Am. J. Physiol. 2004; 267: H2325-2332.
Heylen A, Van Schaftingen E, Hers H G (1982). The stimulation of phosphofructokinase from human erythrocytes by fructose-2,6-bisphosphate. FEBS Lett 143:141-143.
Kaakinen T, Heikkinen J, Dahlbacka S, Alaoja H, Laurila P, Kiviluoma K, Salomaki T, Romsi P, Tuominen H, Biancari F, Lepola P, Nuutinen M, Juvonen T. Fructose-1,6-bisphosphate supports cerebral energy metabolism in pigs after ischemic brain injury caused by experimental particle embolization. Heart Surg. Forum. 2006; 9: E828-835.
Kaakinen T, Naukkarinen A, Tuominen H, Romsi P, Nuutinen M, Biancari F, Juvonen T. Neuronal ultrastructure is preserved by fructose-1,6-bisphosphate after hypothermic circulatory arrest in pigs. J. Thorac Cardiovasc. Surg. 2005; 130: 1475-1476.
Karaca M, Kilic E, Yazici B, Demir S, de la Torre J C (2002). Ischemic stroke in elderly patients treated with a free radical scavenger-glycolytic intermediate solution: a preliminary pilot trial. Neurol Res 24:73-80.
Kelleher J A, Chan P H, Chan T Y, Gregory G A (1995). Energy metabolism in hypoxic astrocytes: protective mechanism of fructose-1,6-bisphosphate. Neurochem Res 20:785-792.
Lammouchi T, Zoghlami F, Ben Slamia L, Grira M, Harzallah M S, Benammou S (2004). Epileptic seizures in non-ketotic hyperglycemia. Neurophysiol Clin 34:183-187.
Lian X Y, Khan F A, Stringer J L. Fructose 1,6 bisphosphate has anticonvulsant activity in models of acute seizures in adult rats, Journal of Neurosci. 2007; 27: 12007-12011.
Lian X Y, Stringer J L (2004). Inhibition of aconitase in astrocytes increases the sensitivity to chemical convulsants. Epilepsy Res. 60:41-52.
Lian X Y, Zhang Z, Stringer J L (2006). Anticonvulsant and neuroprotective effects of ginsenosides in rats. Epilepsy Res 70:244-256.
Liu J, Li L, Wang Y, Chang Y (1998). Clinical study of fructose 1,6-diphosphate on myocardial ischemia/reperfusion injuries. Hunan Yi Ke Da Xue Xue Bao 23:476-478.
Manent J B, Jorquera I, Mazzucchelli I, Depaulis A, Perucca E, Ben-Ari Y, Represa A (2007). Fetal exposure to GABA-acting antiepileptic drugs generates hippocampal and cortical dysplasias. Epilepsia 48:684-693.
Marangos P J, Fox A W, Riedel B J, Royston D, Dziewanawska Z E. Potential therapeutic applications of fructose-1,6-diphosphate. Expert Opin Investig Drugs. 1998; 7: 615-623.
Markov A K, Neely W A, Didlake R H, Terry J 3rd, Causey A, Lehan P H (2000). Metabolic responses to fructose-1,6-diphosphate in healthy subjects. Metabolism 49:698-703.
Markovich D, Murer H. The SLC13 gene family of sodium sulphate/carboxylate cotransporters. Pflugers Arch Eur J. Physiol. 2004; 447: 594-602.
Mueller S G, Trabesinger A H, Boesiger P, Wieser H G (2001). Brain glutathione levels in patients with epilepsy measured by in vivo (1) H-MRS. Neurology 57:1422-1427.
Munger M A, Botti R E, Grinblatt M A, Kasmer R J (1994). Effect of intravenous fructose-1,6-diphosphate on myocardial contractility in patients with left ventricular dysfunction. Pharmacother 14:522-528.
Noh H S, Kim Y S, Lee H P, Chung K M, Kim D W, Kang S S, Cho G J, Choi W S (2003). The protective effect of a ketogenic diet on kainic acid-induced hippocampal cell death in the male ICR mice. Epilepsy Res. 2003; 53:119-128.
Nunes F B, Gaspareto P B, Santos R C V, de Assis M, Graziottin C M, Biolchi V, Filho J C F A, Lunardelli A, Avila L D, Pires M G S, Wachter P H, DeOliveira J R (2003). Intravenous toxicity of fructose-1,6-bisphosphate in rats. Toxicol. Lett. 143:73-81.
Nylen K, Likhodii S, Abdelmalik P A, Clarke J, Burnham W M (2005). A comparison of the ability of a 4:1 ketogenic diet and a 6.3:1 ketogenic diet to elevate seizure thresholds in adult and young rats. Epilepsia 46:1198-1204.
Pasotti C, Nicrosini S, Fiori G (1989). Effects of fructose-1,6-diphosphate in patients with chronic ischemic heart disease. Echocardiographic study. Riv Eur Sci Med Farmacol 11:315-320.
Remington's Pharmaceutical Sciences, 18th Ed. Mack Printing Company, 1990.
Riedel B J, Gal J, Ellis G, Marangos P J, Fox A W, Royston D (2004). Myocardial protection using fructose-1,6-diphosphate during coronary artery bypass graft surgery: a randomized, placebo controlled clinical trial. Anesth Analg 98:20-29.
Ripari P, Pieralisi G (1988). Effects of fructose-1,6-diphosphate on heart rate, ventilation, oxygen consumption and endurance performance. Pharmather 5:249-255.
Rigobello M P, Galzigna L. Pharmacokinetics of fructose-1,6-diphosphate in the rat. IL Farmaco. 1982; 37: 459-462.
Rogido M, Husson I, Bonnier C, Lallemand M C, Merienne C, Gregory G A, Sola A, Gressens P. Fructose-1,6-biphosphate prevents excitotoxic neuronal cell death in the neonatal mouse brain. Dev. Brain Res. 2003; 140: 287-297.
Schwechter E M, Veliskova J, Velisek L (2003) Correlation between extracellular glucose and seizure susceptibility in adult rats. Ann Neurol 53:91-101.
Triguero D, Buciek J, Pardridge W M. Capillary depletion method for quantification of blood-brain barrier transport of circulating peptides and plasma proteins. J. Neurochem. 1990; 54: 1882-1888.
Van Schaftingen E (1987). Fructose-2,6-bisphosphate. Adv Enzymol 59:315-395.
Vexler Z S, Wong A, Francisco C, Manabat C, Christen S, Tauber M, Ferriero D M, Gregory G (2003). Fructose-1,6-bisphosphate preserves intracellular glutathione and protects cortical neurons against oxidative stress. Brain Res 960:90-98.
Wheeler T J, McCurdy J M, denDekker A, Chien S. Permeability of fructose-1,6-bisphosphate in liposomes and cardiac myoctyes. Mol Cell Biochem 2004; 259: 105-114.
Wu G, Fang Y Z, Yang S, Lupton J R, Turner N D (2004). Glutathione metabolism and its implications for health. J Nutr 134:489-492.
Zhao Q, Stafstrom C E, Fu D D, Hu Y, Holmes G L (2004). Detrimental effects of the ketogenic diet on cognitive function in rats. Pediatr Res. 55:498-506.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of preventing one or more seizures in an individual with epilepsy, an individual suspected of having epilepsy, or an individual at risk for developing epilepsy, comprising the step of orally delivering to the individual a therapeutically effective amount of fructose-1,6-bisphosphate.

2. The method of claim 1, wherein the individual is delivered fructose-1,6-bisphosphate at a dosage of 50-150 mg/kg.

3. The method of claim 2, wherein the dosage is 150 mg/kg.

4. The method of claim 1, wherein the individual is provided multiple deliveries of fructose-1,6-bisphosphate.

5. The method of claim 4, wherein the multiple deliveries occur from 12 hours to six days apart.

6. The method of claim 1, further comprising delivering an additional therapy for epilepsy to the individual.

7. The method of claim 6, wherein the additional therapy is a drug, vagus nerve stimulation, surgery, dietary therapy, or a combination thereof.

8. The method of claim 7, wherein the drug is selected from the group consisting of carbamazepine, Carbatrol®, Clobazam, Clonazepam, Depakene®, Depakote®, Depakote ER®, Diastat, Dilantin®, Felbatol®, Frisium, Gabapentin®, Gabitril®, Inovelon®, Keppra®, Klonopin, Lamictal®, Lyrica, Mysoline®, Neurontin®, Oxcarbazepine, Phenobarbital, Phenylek®, Phenyloin, Rufinamide, Sabril, Tegretol®, Tegretol XR®, Topamax®, Trileptal®, Valproic Acid, Zarontin®, Zonegran, and Zonisamide.
